(19) United States
(12) Patent Application Publication
(10) Pub. No.: US 2025/0321203 A1
(43) Pub. Date: Oct. 16, 2025

(54) 2H OR 1T(PRIME) TRANSITION METAL DICHALCOGENIDE, ITS PRODUCTION METHOD AND USES THEREOF

(71) Applicant: CITY UNIVERSITY OF HONG KONG, Kowloon (HK)

(72) Inventors: Zhiyuan ZENG, Kowloon (HK); Liang MEI, Kowloon (HK)

(21) Appl. No.: 19/071,467

(22) Filed: Mar. 5, 2025

Related U.S. Application Data
(60) Provisional application No. 63/562,652, filed on Mar. 7, 2024.

(51) Int. Cl. G01N 27/30; A61B 5/08; G01N 27/416

(52) U.S. Cl. CPC G01N 27/30; A61B 5/082; G01N 27/4166; A61B 2562/029

(57) ABSTRACT

Methods for producing 2H- or 1T′-phase transition metal dichalcogenide (TMD) nanosheets (NSs) includes steps of, (a) discharging a bulk TMD in a lithium battery at small or large current density to produce a lithiated bulk TMD; and (b) subjecting the lithiated bulk TMD to sonification in ethanol or water to exfoliate the lithiated bulk TMD into the 2H- or 1T′-phases TMD NSs; in which discharging the bulk TMD at small and large current densities respectively produce the 2H- and 1T′-phases TMD NSs; and the large current density is about 4 folds of the small current density. Also disclosed herein are devices for sensing humidity. The device includes an electrode characterized by having the 2H- or 1T′-phase TMD NSs produced by the present method.

### CROSS-REFERENCE TO RELATED APPLICATIONS

This application claims priority and the benefit of U.S. Provisional Patent Application No. 63/562,652, filed Mar. 7, 2024, the entirety of which is incorporated herein by reference.

### BACKGROUND OF THE INVENTION

### 1. Field of the Invention

The present invention relates to the production methods of 2H- or 1T′-phase transition metal dichalcogenide (TMD) nanosheets (NSs), and uses of the thus produced 2H- or 1T′-phase TMD NSs for humidity-sensing.

### 2. Description of Related Art

The crystal phase is a pivotal parameter when determining the properties and applications of two-dimensional (2D) transition metal dichalcogenides (TMDs). Group-VI TMDs, such as MoS₂ and WS₂ can exist in diverse phases (2H, 1T or 1T′), depending on the coordination geometries of the transition metal atom. The 1T (octahedral coordination) and 1T′ (distorted octahedral coordination) group-VI TMDs exhibit metallic and semi-metallic properties, respectively, rendering them promising candidates for applications in energy storage, energy conversion and super conductivity. In contrast, the 2H (trigonal prismatic coordination) group-VI TMDs are typically semiconductors with a bandgap of 1-2 eV, making them exceptionally well suited for applications in nanoelectronics and optoelectronics. The preparation of 2D group-VI TMDs (solution-processable TMDs in particular) with a designated phase tailored to intended applications is therefore important, as solution-processable 2D TMDs are compatible with solution-based deposition techniques (such as inkjet printing industrial roll-to-roll coating, drop-casting and spin-coating), facilitating the easy and scalable manufacture of customizable devices. Although such printable 2D group-VI TMD solutions could be prepared via direct liquid exfoliation in solvents and foreign species (for example, tetraalkylammonium ions, sulfate ions and small molecules) intercalation-based liquid exfoliation, the phase of the final 2D products is not adjustable, remaining the same phase as their initial bulk counterparts. Previous studies suggest that 2H-to-1T/1T′ phase transition occurs in group-VI TMDs during lithium-ion (Li⁺) intercalation-based exfoliation, resulting in 2D nanosheets (NSs) with a mixed phase of 2H and 1T/1T′. In theory, this is because the Li⁺ intercalation involves the electron injection from the s orbitals of guest lithium to the d orbitals of the host transition metal atoms to maintain overall charge neutrality. When the electron injection is beyond a certain threshold (in terms of MoS₂ this threshold is 0.29 electrons per formula unit), the stability of the 2H phase group-VI TMDs will be lower than that of the 1T or 1T′ phase, causing the corresponding phase transition. The occurrence of phase transition implies that Li⁺ intercalation-based exfoliation can theoretically become a plausible way for the preparation of solution-processable group-VI 2D TMDs with on-demand phases. Still, this has not yet been truly achieved experimentally, as the switch (on-off) for the phase transition is still unknown.

In view of the above, there exists in the related art a need of a phase-switchable method for the production of 2H- or 1T′-phase TMDs.

### SUMMARY

Embodiments of the present disclosure relate to a method of producing 2H- or 1T′-phases transition metal dichalcogenide (TMD) nanosheets (NSs); a device comprising the thus produced 2H- or 1T′-phases TMD NSs; and a method of detecting humidity by use of the present device.

The first objective of the present disclosure therefore aims to provide a method of producing 2H- or 1T′-phase TMD NSs. The method comprises:
(a) discharging a bulk TMD in a lithium battery at small or large current density to produce a lithiated bulk TMD; and
(b) subjecting the lithiated bulk TMD to sonification in ethanol or water to exfoliate the lithiated bulk TMD into the 2H- or 1T′-phases TMD NSs;
wherein,
discharging the bulk TMD at small and large current densities respectively produce the 2H- and 1T′-phases TMD NSs; and
the large current density is about 4 folds of the small current density.

According to embodiments of the present disclosure, the lithium battery comprises:
an anode made of a lithium foil;
a cathode made of the bulk TMD, carbon black, polyvinylidene difluoride (PVDF) and N-methyl pyrrolidone (NMP); and
an electrolyte consisting of LiPF₆, ethyl carbonate (EC), ethyl methyl carbonate (EMC) and dimethyl carbonate (DMC).

According to some embodiments of the present disclosure, in step (a), the bulk TMD is discharged in the lithium battery at a current density of 0.005 A/g and a cutoff voltage of 0.9V; and in step (b), the lithiated bulk TMD is subjected to sonification in ethanol to exfoliate the lithiated bulk TMD into the 2H-phase TMD NSs.

According to embodiments of the present disclosure, the 2H-phase TMD NSs are 2H-phase WS₂ NSs, 2H-phase TaS₂ NSs, 2H-phase TiS₂ NSs, 2H-phase WSe₂, 2H-phase TaSe₂ NSs, or 2H-phase TiSe₂ NSs. Preferably, the 2H-phase TMD NSs are 2H-phase WS₂ NSs.

According to optional embodiments of the present disclosure, the method further includes,
(c) collecting the 2H-phase TMD NSs of step (b) by centrifugation; and
(d) redispersing the product of step (c) in ethanol to produce a suspension of 2H-phase TMD NSs.

According to other embodiments of the present disclosure, in step (a), the bulk TMD is discharged in the lithium battery at a current density of 0.02 A/g and a cutoff voltage of 0.7V; and in step (b), the lithiated bulk TMD is subjected to sonification in water to exfoliate the lithiated bulk TMD into the 1T′-phase TMD NSs.

According to embodiments of the present disclosure, the 1T′-phase TMD NSs are 1T′-phase MoS₂ NSs, 1T′-phase WS₂ NSs, 1T′-phase TaS₂ NSs, 1T′-phase TiS₂ NSs, 1T′-phase MoSe₂ NSs, 1T′-phase WSe₂ NSs, 1T′-phase TaSe₂ NSs, or 1T′-phase TiSe₂ NSs.

According to optional embodiments of the present disclosure, the method further includes,
(c) collecting the 1T′-phase TMD NSs of step (b) by centrifugation; and
(d) redispersing the product of step (c) in a water to produce a suspension of 1T′-phase TMD NSs.

Accordingly, the second objective of the present disclosure aims at providing a device for detecting humidity. The device comprises an electrode, which comprises a polymeric substrate, and a plurality of 2H- or 1T′-phase TMD NSs produced by the method described above disposed on the polymeric substrate, wherein the electrode has a response time of less than 0.5 second and a recovery time of about 1 second during relative humidity (RH) from about 60% to 75%.

According to embodiments of the present disclosure, the 2H-phase TMD NSs are 2H-phase WS₂ NSs, 2H-phase TaS₂ NSs, 2H-phase TiS₂ NSs, 2H-phase WSe₂, 2H-phase TaSe₂ NSs, or 2H-phase TiSe₂ NSs. Preferably, the 2H-phase TMD NSs are 2H-phase WS₂ NSs.

According to preferred embodiments of the present disclosure, the electrode comprises the plurality of 2H-phase WS₂ NSs disposed on the polymeric substrate, and the electrode has the response time of about 0.48 second and the recovery time of about 0.32 second.

According to certain embodiments of the present disclosure, the 1T′-phase TMD NSs are 1T′-phase MoS₂ NSs, 1T′-phase WS₂ NSs, 1T′-phase TaS₂ NSs, 1T′-phase TiS₂ NSs, 1T′-phase MoSe₂ NSs, 1T′-phase WSe₂ NSs, 1T′-phase TaSe₂ NSs, or 1T′-phase TiSe₂ NSs.

According to preferred embodiments of the present disclosure, the electrode comprises the plurality of 1T′-phase WS₂ NSs disposed on the polymeric substrate, and the electrode has the response time of about 0.3 second and the recovery time of about 1.2 second.

The third aspect of the present disclosure thus is directed to a method of determining humidity in a gas exhaled from the nose or the mouth of a subject. The method includes steps of:
(a) contacting the exhaled gas with the present device;
(b) measuring the intensity of a current generated from the electrode upon contacting with the exhaled gas; and
(c) determining the humidity in the exhaled gas by interpolating the measured intensity of step (b) from a standard current intensity and relativity humidity (RH) plot;
wherein,
the standard current intensity and RH plot is made by plotting a plurality of known current intensities of the electrode verses their corresponding RH levels.

According to some embodiments of the present disclosure, the device comprises an electrode, which has a plurality of 2H-phase TMD NSs disposed on a polymeric substrate, and the electrode has a response time of about 0.48 second and a recovery time of about 0.32 second.

According to other embodiments of the present disclosure, the device comprises an electrode, which has a plurality of 1T′-phase TMD NSs disposed on a polymeric substrate, and the electrode has a response time of about 0.3 second and a recovery time of about 1.2 second.

In all embodiments of the present disclosure, the subject is a human.

Other and further embodiments of the present disclosure are described in more detail below.

### BRIEF DESCRIPTION OF THE DRAWINGS

The disclosure will become more fully understood from the detailed description and the drawings given herein below for illustration only, and thus does not limit the disclosure, wherein:

FIG. 1 is a flow chart illustrating steps of a method 10 for producing 2H- or 1T′-phases TMD NSs in accordance with one preferred embodiment of the present disclosure;

FIG. 2a is visible near-infrared absorption spectra of exfoliated 2H- and 1T′-phase WS₂ NSs solutions, in which A, B and C indicate three absorption peaks in the exfoliated 2H-WS₂, and the inset are photographs of exfoliated 2H- and 1T′-phase WS₂ NSs solutions;

FIG. 2b is PL spectra of exfoliated 2H- and 1T′-phase WS₂ in accordance with one preferred embodiment of the present disclosure;

FIG. 2c is Raman spectra of bulk WS₂ and exfoliated 2H- and 1T′-phase WS₂ in accordance with one preferred embodiment of the present disclosure;

FIG. 2d is XPS W4f spectra of bulk WS₂ and exfoliated 2H- and 1T′-phase WS₂ in accordance with one preferred embodiment of the present disclosure;

FIG. 3a is the I-RH curve of the present 2H-WS₂ humidity sensor, and the inset is the enlarged curve of low RH levels between 15% to 50%;

FIG. 3b is the dynamic humidity response and recovery measurements of the 2H-WS₂ humidity sensor of FIG. 3a under different RH levels;

FIG. 3c is the dynamic humidity response and recovery curves of the 2H-WS₂ humidity sensor of FIG. 3a from 60% RH to 75% RH;

FIG. 3d is the amplified response and recovery curve from FIG. 3c, in which τᵣᵢₛₑ and τ_{fall} represent response and recovery times;

FIG. 3e is the response and recovery curve of the 1T′-WS₂ humidity sensor from 60% RH to 75% RH;

FIG. 3f is the amplified response and recovery curve from FIG. 3e, in which τᵣᵢₛₑ and τ_{fall} represent response and recovery times;

FIG. 4a illustrates the nasal breath monitoring curve of 2H-WS₂ humidity sensor in accordance with one preferred embodiment of the present disclosure;

FIG. 4b illustrates the quantitative measurements between finger distance and device current under a bias of 10 V, the inset shows the finger approaching the device; and

FIG. 4c is the dynamic current curves of the WS₂ humidity sensor as the finger approaches in accordance with one preferred embodiment of the present disclosure.

### DETAILED DESCRIPTION

Detailed descriptions and technical contents of the present disclosure are illustrated below in conjunction with the accompanying drawings. However, it is to be understood that the descriptions and the accompanying drawings disclosed herein are merely illustrative and exemplary and not intended to limit the scope of the present disclosure.

### 1. Methods of Producing 2H- or 1T′-TMD NSs

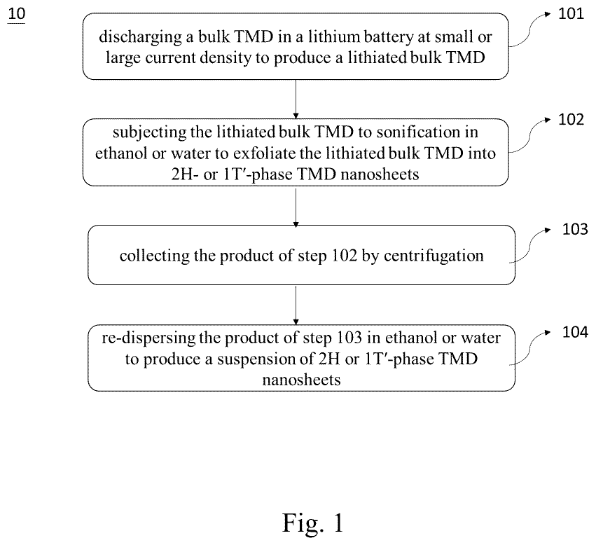

The first objective of the present disclosure is directed to a method of producing 2H- or 1T′-phases TMD NSs by electrochemical lithium intercalation at small or large current density, followed by solution-based exfoliation, detail steps are described in the flowchart in FIG. 1.

To this purpose, a lithium battery for performing the present method is constructed. Specifically, a slurry containing a bulk TMD powders, carbon black, and polyvinylidene difluoride (PVDF) are mixed with N-methyl pyrrolidone (NMP) to give a mixture, which is fabricated into a cathode. The cathode is assembled with an anode (i.e., a lithium foil), and an electrolyte into the lithium battery, according to preferred embodiments of the present disclosure, the electrolyte consists of LiPF₆ dissolved in a mixture of ethyl carbonate (EC), ethyl methyl carbonate (EMC) and dimethyl carbonate (DMC) in a 1:1:1 volume ratio. Once the battery is constructed, a potential difference is applied to the battery to intercalate lithium ions into the cathode.

To initiate the present method, the lithium battery is discharged at small or large current density to produce a lithiated bulk TMD (FIG. 1 step 101). According to embodiments of the present disclosure, discharging the lithium battery at small and large current densities may lead to the production of the 2H- and 1T′-phases TMD NSs, respectively, in which the large current density is about 4 folds of the small current density.

After electrochemical lithium intercalation, the lithiated bulk TMD is then subjected to sonification in ethanol or water to exfoliate the lithiated bulk TMD into the desired 2H- or 1T′-phases TMD NSs (FIG. 1, step 102).

In some embodiments, in step 101, the bulk TMD is discharged in the lithium battery at a current density of 0.005 A/g and a cutoff voltage of 0.9V; and in step 102, the lithiated bulk TMD is subjected to sonification in ethanol to exfoliate the lithiated bulk TMD into the 2H-phase TMD NSs. Examples of 2H-phase TMD NSs that may be produced by the present method include, but are not limited to, 2H-phase WS₂ NSs, 2H-phase TaS₂ NSs, 2H-phase TiS₂ NSs, 2H-phase WSe₂, 2H-phase TaSe₂ NSs, or 2H-phase TiSe₂ NSs. Preferably, the 2H-phase TMD NSs are 2H-phase WS₂ NSs.

In other embodiments, in step 101, the bulk TMD is discharged in the lithium battery at a current density of 0.02 A/g and a cutoff voltage of 0.7V; and in step 102, the lithiated bulk TMD is subjected to sonification in water to exfoliate the lithiated bulk TMD into the 1T′-phase TMD NSs. Examples of the 1T′-TMD NSs that may be produced by the present method include, but are not limited to, 1T′-phase MoS₂ NSs, 1T′-phase WS₂ NSs, 1T′-phase TaS₂ NSs, 1T′-phase TiS₂NSs, 1T′-phase MoSe₂ NSs, 1T′-phase WSe₂ NSs, 1T′-phase TaSe₂ NSs, or 1T′-phase TiSe₂ NSs. According to preferred embodiments of the present disclosure, the 1T′-TMD NSs are 1T′-WS₂ NSs.

Optionally, the thus produced 2H- or 1T′-phases TMD NSs are subsequently collected via centrifugation (FIG. 1, step 103), followed by re-dispersing the collected 2H- or 1T′-phases TMD NSs in ethanol or water to give a suspension of 2H- or 1T′-phase TMD NSs (FIG. 1, step 104). The suspension of 2H- or 1T′-phase TMD NSs may be used to construct devices (e.g., electrodes) for further applications, such as humidity-sensing.

### 2. Humidity-Sensing Device

The present 2H- or 1T′-phase TMD NSs could absorb water molecules, thus are suitable for sensing atmospheric humidity. Accordingly, the suspension of 2H- or 1T′-phase TMD NSs described above is used to fabricate an electrode suitable for constructing a humidity-sensing device.

To this purpose, the suspension of 2H- or 1T′-phase TMD NSs described above is drop-cast onto a polymeric substrate, which is allowed to dry at room temperature. Then, the substrate is sputter-coated with a thin layer of a conductive material, typically, a metal, such as gold (Au), palladium (Pd), an alloy of Au/Pd and the like. Examples of the polymeric substrate suitable for use in the present disclosure include, but are not limited to, nylon, polyethylene terephthalate (PET), polyvinylidene difluoride (PVDF), and the like. According to preferred embodiments of the present disclosure, varying volumes of the suspension of 2H- or 1T′-phase TMD NSs are filtered over PET, and then sputter-coated with Au (about 200 nm in thickness), thereby producing the desired electrode.

Accordingly, the second objective of the present disclosure is to provide a device suitable for detecting humidity, such as the breath humidity exhaled from the nose or the mouth of a subject. The device is characterized by having the present electrode described above. According to embodiments of the present disclosure, the electrode comprises a plurality of 2H- or 1T′-phases TMD NSs produced by the method described above in Section 1 of this paper, and may detect humidity with a response time of less than 1 second and a recovery time of about 1 second from relative humidity (RH) 60% to 75%.

In some embodiments, the device comprises an electrode characterized by having a plurality of the 2H-phase TMD NSs (e.g., 2H-phase WS₂ NSs) produced by the present method, and the electrode may detect humidity with a response time of about 0.48 second and a recovery time of about 0.32 second. In other embodiments, the device comprises an electrode characterized by having a plurality of the 1T′-phase TMD NSs (e.g., 1T′-phase WS₂ NSs) produced by the present method, and may detect humidity with a response time of about 0.3 second and a recovery time of about 1.2 second.

### 3. Methods for Sensing Humidity

The present disclosure thus also encompasses a method of sensing humidity in a gas exhaled from the nose or the mouth of a subject, with the aid of the device described in Section 2 of this paper.

According to embodiments of the present disclosure, the present device may be placed underneath the nostrils of the subject, or in front of the mouth of the subject, so as to catch the water molecules present in the gas exhaled from the nose or the mouth of the subject. The method thus includes steps of:
(a) contacting the exhaled gas with the present device;
(b) measuring the intensity of a current generated from the electrode upon contacting with the exhaled gas; and
(c) determining the humidity in the exhaled gas by interpolating the measured intensity of step (b) from a standard current intensity and relativity humidity (RH) plot;
wherein,
the standard current intensity and RH plot is made by plotting a plurality of known current intensities of the electrode verses their corresponding RH levels.

According to some embodiments of the present disclosure, the electrode has a plurality of 2H-phase WS₂ NSs disposed on a PET substrate, and the response and recovery times are respectively about 0.48 second and about 0.32 second during RH level from about 60% to about 75%.

According to other embodiments of the present disclosure, the electrode has a plurality of 1T′-phase WS₂ NSs disposed on a PET substrate, and the response and recovery times are respectively about 0.3 second and about 1.2 second during RH level from about 60% to about 75%.

According to all embodiments of the present disclosure, the subject is human.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

### Material and Methods

Synthesis of 2H- or 1T′-phase WS₂ NSs. The 2H- or 1T′-WS₂ NSs were produced by electrochemical lithium exfoliation-based intercalation protocol in a coin cell setup using a copper foil-coated bulk WS₂ as the cathode (weighing 5 mg), lithium foil as the anode and an electrolyte solution comprising lithium hexafluorophosphate (LiPF₆) dissolved in a mixture of ethylene carbonate (EC), dimethyl carbonate (DMC) and ethyl methyl carbonate (EMC) in a 1:1:1 volume ratio. A galvanostatic discharge was applied to initiate the Li⁺ intercalation of the bulk WS₂. Upon completion of the intercalation process, the resulting lithium-intercalated WS₂, (LiₓWS₂) was carefully extracted from the coin cell and subjected to sonication in appropriate solvents to achieve exfoliation. To produce 2H-WS₂ NSs, the coin cell was discharged at 0.005 A/g and a cutoff voltage of 0.9 V, whereas for 1T′-WS₂ NSs, the coin cell was discharged at 0.02 A/g and 0.7 V. In addition, 2H-WS₂ NSs were pre-pared by sonication in ethanol, whereas 1T′-WS₂ NSs were fabricated through sonication in deionized water. Following the sonication step, both the 2H- and 1T′-WS₂ suspensions underwent centrifugation and multiple washing steps before being ultimately redispersed in a solvent for further characterization and device fabrication.

### Fabrication of WS₂-Based Device

The PET substrate (1.5×1.5 cm²) underwent a thorough cleaning process in an ultrasonic bath. It was sequentially cleaned with detergent water, acetone, deionized water and isopropyl alcohol, with each cleaning step lasting for 15 min. After this cleaning procedure, an ethanol dispersion of exfoliated WS₂ NSs (32 mg/L) was drop-cast on to the PET substrate and allowed to dry at room temperature. Finally, 200-nm-thick Au electrodes were sputtered onto the PET substrate using a QUORUM #Q150TS Dual target sputtering system, employing a designed metal mask.

In the fabrication of the WS₂-based sensor array, the PET substrates underwent the previously described cleaning process. Following this, a circuit design based on Au was meticulously created using the QUO-RUM #Q150TS Dual target sputtering system, with the aid of a precisely designed metal mask. Subsequently, a custom metal mask with a rectangular array pattern was accurately affixed onto the as-fabricated circuit. This attachment was made using specific counter points to ensure accuracy. An ethanol dispersion of WS₂ NSs (32 mg/L) was then drop-cast onto the substrate. Afterwards, the mask was carefully removed once the solution had completely dried, leaving the WS₂ NSs in the desired pattern on the substrate.

### Humidity-Sensing Tests

The humidity-sensing performance was evaluated within a custom-made sealed container. Various humidity levels were precisely controlled by adjusting the mass flow of dry and moistened argon gas that was introduced into the container. The chip carrier was connected to external measurement equipment (Keysight BISOOA Semiconductor Analyze) for conducting the tests. For the breathing test, a humidity sensor was placed under the nose or on masks to monitor exhaled gas humidity at different breathing rates. In the humidity sensing of the finger approach experiment, the sensor was positioned horizontally, and a finger was gradually brought closer from above. For touchless localization, the finger was held at a fixed height above the sensor array, and the current of each sensor was measured. In the voice recognition test, the sensor was placed 5 cm in front of the subject's mouth, and current changes were recorded as they pronounced English words with different lengths. All sensor currents were recorded in real time using a Keysight BISO0A Semiconductor Analyzer.

### Example 1: Fabrication and Characterization of 2H- or 1T′-TMD NSs

### 1.1 2H- or 1T′-WS₂ NSs

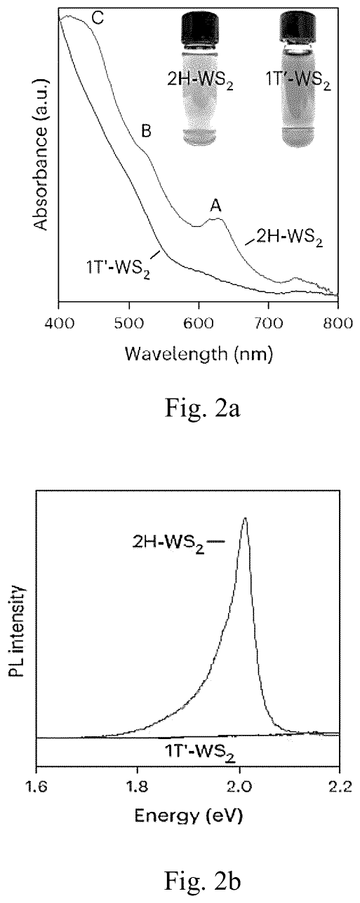

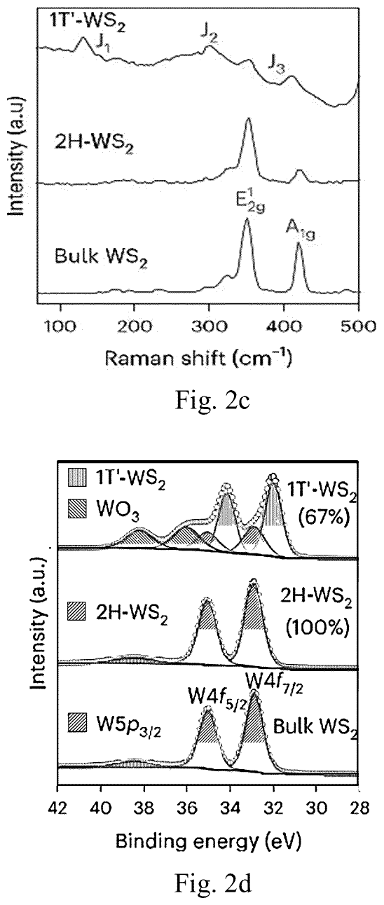

In this example, 2H- or 1T′-phase WS₂ NSs were respectively produced in accordance with procedures described in the “Materials and Methods” section. Specifically, a lithium coin cell comprising a cathode made of a bulk WS₂ was discharged at small (0.005 A/g, 0.9 V) or large (0.02 A/g, 0.7 V) current density and exfoliated to produce 2H- or 1T′-phases WS₂ NSs, which were characterized by UV-Vis, Raman, and X-ray photoelectron spectroscopy. Results are illustrated in FIG. 2.

The small current density-driven Li⁺ intercalation-based exfoliated WS₂ NSs dispersion exhibited a light-green color, signifying partial absorption in the visible range, as verified via the ultraviolet-visible absorption spectrum (FIG. 2a), which confirmed the formation of semiconducting 2H-phase WS₂ NSs under a small discharging current density. Conversely, the large current density exfoliated WS₂ NSs dispersion showed a black color, indicating the complete and featureless absorption in the visible range (FIG. 2a), marking the emergence of phase transition and the formation of metallic 1T/1T′-phase WS₂ NSs under a large discharging current density.

Photoluminescence (PL), Raman spectroscopy and X-ray photoelectron spectroscopy (XPS) further confirmed the above phase analysis. Prominent PL appeared in small current density exfoliated WS₂ NSs (FIG. 2b), signifying its semiconducting nature (2H-phase). Only two dominant A comparison (the in-plane phonon E¹_{2g} at 350.3 cm⁻¹ and the out-of-plane phonon A_{1g} modes at 419.2 cm⁻¹, corresponding to 2H-WS₂) appeared in the Raman spectroscopy of small current density exfoliated WS₂ NSs (FIG. 2c), indicating their 2H-phase. By contrast, the J₁, J₂, and J₃ peaks located at low frequency regions were observed for large current density exfoliated WS₂ NSs (FIG. 2c), indicative of the 1T′-phase. The yields of 2H-WS₂ (under small current density) and 1T′-WS₂ (under large current density) NSs were ˜100% and ˜67%, respectively, as confirmed by the deconvolution of the W 4f regions of exfoliated WS₂ in the XPS spectrum (FIG. 2d).

The phases of exfoliated WS₂ NSs were further confirmed by aberration-corrected annular dark-field scanning transmission electron microscopy (ADF-STEM). The ADF-STEM image of the small current density exfoliated WS₂ NSs showed a hexagonal lattice structure of individual W and S atoms, confirming the 2H-phase, whereas the one-dimensional zigzag chains of W atoms dominated the entire image of the large current density exfoliated WS₂ NSs, indicative of the 1T′-phase. Notably, a small area of 2H-phase was also observed in the image, suggesting the co-existence of 1T′- and 2H-phases in large current density exfoliated WS₂ NSs, and 1T′-phase was dominant. Sulfur vacancies were also clearly observed in the ADF-STEM images of the prepared 2H- and 1T′-WS₂ NSs (data not shown), and the electron paramagnetic resonance (EPR) results indicated that the sulfur vacancies in 2H-WS₂ NSs were more abundant than those in 1T′-WS₂ NSs (data not shown).

Transmission electron microscopy (TEM) revealed that the exfoliated 2H- and 1T′-WS₂ NSs had a lateral size ranging from 300 to 600 nm and 100 to 200 nm, respectively (data not shown). Large quantities of 2H- and 1T′-phase WS₂ NSs were successfully prepared as confirmed by scanning electron microscope (SEM) images. Atomic force microscopy (AFM) images showed that the thickness of the 2H-phases WS₂ NSs was 1.8 nm, while that of 1T′-phases WS₂ NSs was 1.2 nm, indicating the formation of bilayer 2H- and monolayer 1T′-WS₂ NSs.

### 1.2 2H- or 1T′-WSe₂ NSs

Phase-switchable preparation of WSe₂ was also successfully produced and characterized in accordance with similar procedures described in Example 1.1 (data not shown).

### 1.3 1T′-MoS₂ NSs

It was found that the present phase-switchable preparation method was not suitable for MoS₂; only 1T′-MoS₂ NSs could be prepared (data not shown).

### Example 2: Measurement of Humidity by Use of a Device Fabricated from the 2H- or 1T′-WS₂ NSs of Example 1

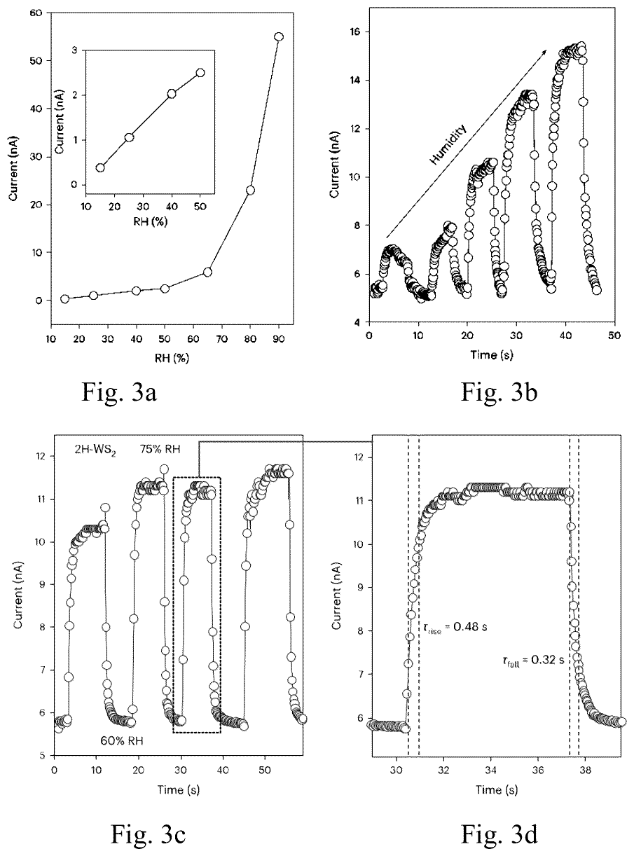

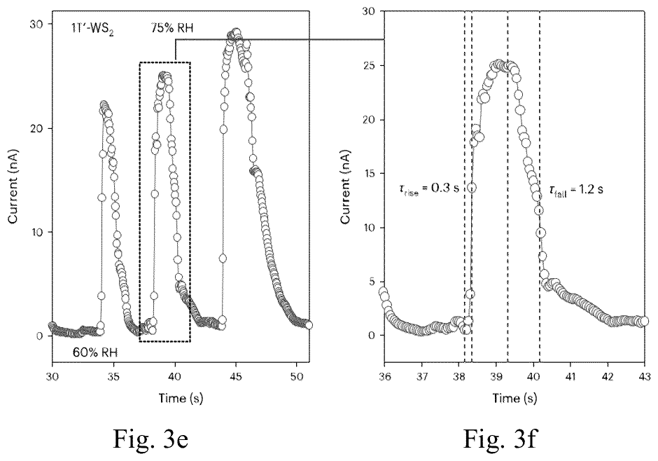

In this example, the 2H- or 1T′-WS₂ NSs of Example 1 were used to fabricate humidity sensors in accordance with the procedures described in the “Materials and Methods” section. Briefly, the exfoliated 2H- or 1T′-WS₂ NSs of Example 1 were drop-casting onto a PET substrate. After drying, Au electrodes were sputtered onto the 2H- or 1T′-WS₂ NSs, the thus fabricated device was then used to measure humidity at different relative humidity (RH) conditions. Results are illustrated in FIGS. 3a to 3f.

As shown in FIG. 3a, the current of the device at a bias of 10V increased by nearly two orders of magnitude with an increase in the RH level from 15% to 90%, suggesting excellent humidity-sensing capability of the fabricated device. Notably, the 2H-WS₂ humidity sensor exhibited a stronger response when the applied RH exceeded 65%, signifying that such a humidity sensor was well suited for applications requiring detection in high-humidity environments, such as breath rate monitoring and non-touch positioning interfaces. The dynamic humidity response and recovery measurements of the 2H-WS₂ humidity sensors are shown in FIG. 3b, demonstrating excellent selectivity and repeatability when responding to different levels of RH. Response and recovery times are crucial parameters that determine the sensitivity of humidity sensors. FIG. 3c depicts the repeatable dynamic humidity response of the 2H-WS₂ humidity sensors from RH 60% to RH 75%, demonstrating ultrafast response and recovery times below Is. An enlarged I-T (I, current; T, time) curve (FIG. 3d) revealed that the response and recovery times were respectively 0.48 s and 0.32 s for 2H-WS₂ humidity sensor. In comparison, these values were 0.30 s and 1.20 s for the 1T′-WS₂ humidity sensor tested under the same conditions (FIGS. 3e and 3f). Compared with the 2H-WS₂ humidity sensor, the 1T′-WS₂ humidity sensor exhibited a faster response but a slower recovery time. This difference might be attributed to the fact that 1T′-WS₂ possessed more negative adsorption energy towards water molecules than 2H-WS₂ (data not shown), implying that water molecules were more easily adsorbed on 1T′-WS₂. Conversely, water molecules were more challenging to desorb from the surface of 1T′-WS₂, resulting in a longer recovery time.

When comparing the response and recovery times with other reported humidity sensors, both the present fabricated 2H- and 1T′-WS₂ sensors demonstrated the fastest recovery and response times (data not shown). The origin of short recovery and response time was due to the ultrathin structure of the exfoliated WS₂ NSs and the presence of abundant S defects. Specifically, the present 1T′-WS₂ and 2H-WS₂ NSs, being monolayer and bilayer, respectively, were significantly thinner than other humidity sensor materials (data not shown). The present 1T′-WS₂ and 2H-WS₂ materials possessed an exceptionally high surface-to-volume ratio, which resulted in a greater number of atoms being exposed on the surface, enhancing the interaction with water molecules. This high surface area facilitated increased adsorption of water molecules, leading to substantially increased changes in electrical properties and, consequently, higher sensitivity to humidity variations. Hence, the minimal thickness of these 2D materials enabled rapid adsorption and desorption of water molecules, contributing to faster response and recovery times. Furthermore, the present 1T′-WS₂ and 2H-WS₂ NSs possessed abundant sulfur defects, which enhanced conductivity and subsequently shortened the response time and improved the recovery speed. These factors sufficiently explained the short response and recovery times observed in the present 1T′-WS₂ and 2H-WS₂ NSs. Based on the relatively faster response and recovery speed, 2H-WS₂ NSs were selected for the following humidity-sensing demonstration.

### Example 3: Application of Devices Fabricated from the 2H- or 1T′-WS₂ NSs of Example 1

### 3.1 Breath Monitoring Device

The mechanical properties of the 2H-WS₂ humidity sensor were examined by analyzing its response current under RH 65% after subjecting it to different cycles of bending. The WS₂ humidity sensor demonstrated exceptional flexibility, with the response current remaining nearly unchanged even after 1,300 cycles of bending (data not shown). This resilience could be attributed to the use of exfoliated WS₂ NSs, which were intrinsically flexible ultrathin 2D materials with a high fracture strain of approximately 25-30%. These properties significantly contributed to the stability of the fabricated devices. The present WS₂ humidity sensor was thus used for breath monitoring, a critical aspect of diagnosing and treating slowly progressing diseases with few early indicators (for example cancer, diabetes and sleep apnoea-hypopnoea syndrome). The WS₂ humidity sensor was easily attached under the human nose to demonstrate nasal breath monitoring.

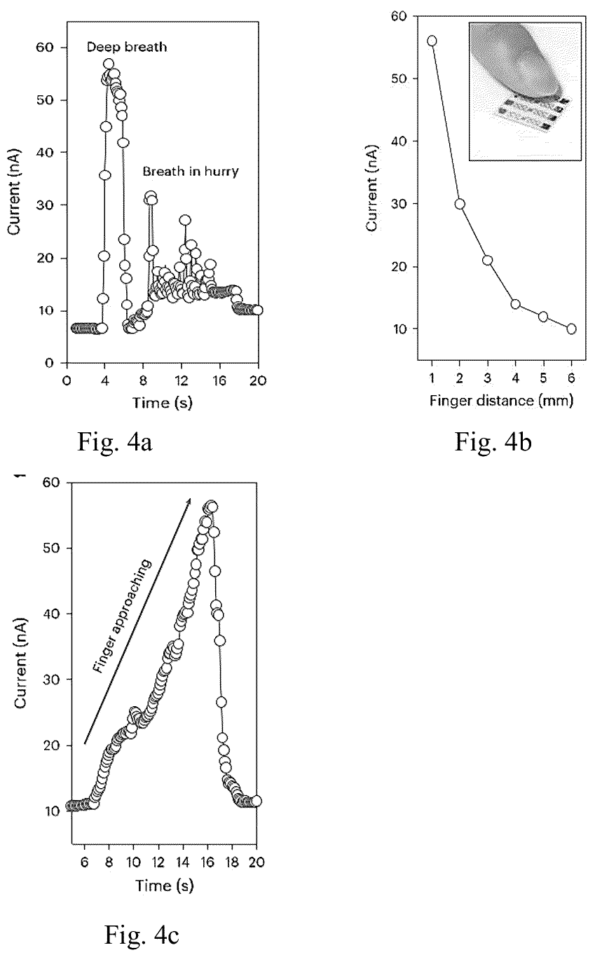

As shown in FIG. 4a, the device's current value simultaneously varied in response to changes in breath rate. During the 3-7 s of measurement, the tested subject took a deep breath and exhaled a high RH level flow, resulting in a significant rise and fall in current. Subsequently, the subject breathed at a quick and uneven rate within 8 s, leading to low-amplitude and high-frequency fluctuations in the device's current. These results demonstrated that the ultrafast response and recovery properties of the present WS₂ humidity sensor could effectively support precise real-time breath monitoring. In addition, the WS₂ humidity sensor could be employed to test the protective performance of various types of mask, which holds significant importance, such as during the coronavirus disease 2019 epidemic.

### 3.2 Speech Recognition Device

A prototype speech recognition device has also been developed. When a person speaks, the pronunciation of different words leads to variations in the concentration of exhaled water vapor. The ultrafast response and recovery of the WS₂-based humidity sensor allowed it to detect real-time changes in breath humidity associated with spoken words (data not shown). Consequently, this generated characteristic current curves corresponding to each word. The characteristic current curves were recorded by the device for various spoken words, including ‘Hi’, ‘people’, ‘thank you’, ‘you are welcome’, ‘question’, ‘unbelievable’ and ‘beautiful’. These current response curves exhibit unique and distinct characteristics. A comparison with high repeatability, highlighting the device's significant potential for voice recognition applications.

3.3 Device with Touchless Interface

The long-range manipulation of devices offers promising potential for future electronics, particularly in applications like non-touch switches and touchless localization interfaces. Moisture detection could serve as a compelling stimulus signal to enable these innovative applications. The high sensitivity and rapid responsiveness of the present fabricated WS₂ humidity sensor are more than capable of meeting the requirements for real-time touchless localization interfaces. As illustrated in FIG. 4b (inset), the human finger served as the moisture source. Quantitative measurements under a bias of 10 V in FIG. 4b demonstrated an exponential relationship between finger distance and the response current of a single WS₂ humidity sensor. The sensor began to show a slight response at a vertical height of 6 mm that gradually increased as the finger approached. FIG. 4c reveals the dynamic current of the device in response to an approaching finger in an indoor environment. Benefitting from the ultrafast response and recovery time, the device's current increased as the finger approached without any hysteresis. Furthermore, the superior stability of the WS₂ humidity sensor was confirmed by exposing it to the indoor environment (65% RH, room temperature) for 96 h (data not shown).

Prompted by the excellent response capabilities, a touchless localization interface could be realized using the WS₂ humidity sensor array. A 5×5 WS₂ humidity sensor array on a PET substrate was produced accordingly. The thus fabricated touchless localization interface retained its flexibility. Even under bending conditions, the response and recovery properties remained nearly unchanged compared with the flat condition (data not shown). It was found the relative position and depth of two fingers could be successfully distinguished using the WS₂-based localization interface (data not shown). The current distribution map revealed the location of the two fingers, with colors indicating the vertical distance between the fingers and the sensor array. By incorporating the information of the location and depth of the fingers in a three-dimensional (3D) cuboid, a reconstructed 3D model that closely resembled the actual positions of the two fingers could be achieved. These results suggested that the WS₂-based localization interface could be employed to reconstruct the spatial distribution of subjects on the basis of moisture stimulus signals. Furthermore, due to its fast response and recovery properties, real-time location monitoring can be realized using the present touchless localization interface.

In summary, the present disclosure demonstrated that electric switch of phase transition during the electrochemical Li⁺ intercalation-based exfoliation, achieving phase-switchable exfoliation of solution-processable WS₂ mono- or bilayers from their bulk counterparts. Specifically, the present disclosure demonstrates that Li⁺ intercalation under a small discharge current density will cause the formation of semiconducting 2H-WS₂ bilayers. Conversely, a large discharge current density will lead to 2H-to-1T/1T′ phase transition and thereby produces semi-metallic 1T′-WS₂ monolayers.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. A method of producing 2H- or 1T′-phase TMD nanosheets comprising:
(a) discharging a bulk TMD in a lithium battery at small or large current density to produce a lithiated bulk TMD; and
(b) subjecting the lithiated bulk TMD to sonification in ethanol or water to exfoliate the lithiated bulk TMD into the 2H- or 1T′-phases TMD nanosheets;
wherein,
discharging the bulk TMD at small and large current densities respectively produce the 2H- and 1T′-phases TMD nanosheets; and
the large current density is about 4 folds of the small current density.

2. The method of claim 1, wherein the lithium battery comprises:
an anode made of a lithium foil;
a cathode made of the bulk TMD, carbon black, polyvinylidene difluoride (PVDF) and N-methyl pyrrolidone (NMP); and
an electrolyte consisting of LiPF₆, ethyl carbonate (EC), ethyl methyl carbonate (EMC) and dimethyl carbonate (DMC).

3. The method of claim 2, wherein
in step (a), the bulk TMD is discharged in the lithium battery at a current density of 0.005 A/g and a cutoff voltage of 0.9V; and
in step (b), the lithiated bulk TMD is subjected to sonification in ethanol to exfoliate the lithiated bulk TMD into the 2H-phase TMD nanosheets.

4. The method of claim 3, wherein the 2H-phase TMD nanosheets are 2H-phase WS₂ nanosheets, 2H-phase TaS₂ nanosheets, 2H-phase TiS₂ nanosheets, 2H-phase WSe₂, 2H-phase TaSe₂ nanosheets, or 2H-phase TiSe₂ nanosheets.

5. The method of claim 3, further comprising:
(c) collecting the 2H-phase TMD nanosheets of step (b) by centrifugation; and
(d) redispersing the product of step (c) in ethanol to produce a suspension of 2H-phase TMD nanosheets.

6. The method of claim 2, wherein
in step (a), the bulk TMD is discharged in the lithium battery at a current density of 0.02 A/g and a cutoff voltage of 0.7V; and
in step (b), the lithiated bulk TMD is subjected to sonification in water to exfoliate the lithiated bulk TMD into the 1T′-phase TMD nanosheets.

7. The method of claim 6, wherein the 1T′-phase TMD nanosheets are 1T′-phase MoS₂ nanosheets, 1T′-phase WS₂ nanosheets, 1T′-phase TaS₂ nanosheets, 1T′-phase TiS₂ nanosheets, 1T′-phase MoSe₂ nanosheets, 1T′-phase WSe₂ nanosheets, 1T′-phase TaSe₂ nanosheets, or 1T′-phase TiSe₂ nanosheets.

8. The method of claim 7, further comprising:
(c) collecting the 1T′-phase TMD nanosheets of step (b) by centrifugation; and
(d) redispersing the product of step (c) in a water to produce a suspension of 1T′-phase TMD nanosheets.

9. A device for sensing humidity comprising an electrode having a polymeric substrate, and a plurality of 2H- or 1T′-phases TMD nanosheets produced by the method of claim 1 disposed on the polymeric substrate, wherein the electrode has a response time of less than 0.5 second and a recovery time of about 1 second during relative humidity (RH) from about 60% to 75%.

10. The device of claim 9, wherein,
in step (a), the bulk TMD is discharged in the lithium battery at a current density of 0.005 A/g and a cutoff voltage of 0.9V; and
in step (b), the lithiated bulk TMD is subjected to sonification in ethanol to exfoliate the lithiated bulk TMD into the 2H-phase TMD nanosheets.

11. The device of claim 10, wherein the 2H-phase TMD nanosheets are 2H-phase WS₂ nanosheets, 2H-phase TaS₂ nanosheets, 2H-phase TiS₂ nanosheets, 2H-phase WSe₂, 2H-phase TaSe₂ nanosheets, or 2H-phase TiSe₂ nanosheets.

12. The device of claim 11, wherein the 2H-phase TMD nanosheets are the 2H-phase WS₂ nanosheets, and the electrode has the response time of about 0.48 second and the recovery time of about 0.32 second.

13. The device of claim 9, wherein,
in step (a), the bulk TMD is discharged in the lithium battery at a current density of 0.02 A/g and a cutoff voltage of 0.7V; and
in step (b), the lithiated bulk TMD is subjected to sonification in water to exfoliate the lithiated bulk TMD into the 1T′-phase TMD nanosheets.

14. The device of claim 13, wherein the 1T′-phase TMD nanosheets are 1T′-phase MoS₂ nanosheets, 1T′-phase WS₂ nanosheets, 1T′-phase TaS₂ nanosheets, 1T′-phase TiS₂ nanosheets, 1T′-phase MoSe₂ nanosheets, 1T′-phase WSe₂ nanosheets, 1T′-phase TaSe₂ nanosheets, or 1T′-phase TiSe₂ nanosheets.

15. The device of claim 14, wherein the 1T′-phase TMD nanosheets are the 1T′-phase WS₂ nanosheets, and the electrode has the response time of about 0.3 second and the recovery time of and 1.2 second.

16. A method of determining humidity in a gas exhaled from the nose or the mouth of a subject comprising:
(a) contacting the exhaled gas with the device of claim 9;
(b) measuring the intensity of a current generated from the electrode upon contacting with the exhaled gas; and
(c) determining the humidity in the exhaled gas by interpolating the measured intensity of step (b) from a standard current intensity and relativity humidity (RH) plot;
wherein,
the standard current intensity and RH plot is made by plotting a plurality of known current intensities of the electrode verses their corresponding RH levels.

17. The method of claim 16, wherein the electrode has a plurality of 2H-phase TMD nanosheets disposed on the polymeric substrate, and the response time of about 0.48 second and the recovery time of about 0.32 second.

18. The method of claim 16, wherein the electrode has a plurality of 1T′-phase TMD nanosheets disposed on the polymeric substrate, and the response time of about 0.3 second and the recovery time of about 1.2 second.

19. The method of claim 16, wherein the subject is a human.
